(19) United States
(12) Patent Application Publication
(10) Pub. No.: US 2023/0277351 A1
(43) Pub. Date: Sep. 7, 2023

(54) ADJUSTABLE ANKLE JOINT DISTRACTION DEVICE

(71) Applicant: RK Innovations, LLC, Fort Collings, CO (US)

(72) Inventor: Reginald Kellum, Fort Collins, CO (US)

(21) Appl. No.: 18/178,387

(22) Filed: Mar. 3, 2023

Related U.S. Application Data
(60) Provisional application No. 63/268,843, filed on Mar. 3, 2022.

(51) Int. Cl. A61F 5/01

(52) U.S. Cl. CPC A61F 5/0111; A61F 2005/0132

(57) ABSTRACT

Existing offloading braces rely on a fixed suspension of the user's afflicted ankle joint, thereby offloading it. However, by suspending the ankle joint and replacing its contact with the ground with another device, one of the user's legs is elongated with reference to the other non-injured leg. This can cause issues with the user's non-injured leg over time as the user adjusts their gait to compensate. Further, by suspending the ankle joint, any articulation previously offered by the ankle joint in walking is forfeit. Such offloading braces can yield an unnatural “peg-like” quality to the user's gait. The presently disclosed adjustable ankle joint distraction devices distract a user's ankle joint, while preserving a sensation of contact of the user's foot with the ground and articulation of the user's ankle joint with reference to the ground.

### CROSS-REFERENCE TO RELATED APPLICATIONS

The present application claims benefit of priority to U.S. Provisional Patent Application No. 63/268,843 entitled “Adjustable Ankle Joint Distraction Device” and filed on Mar. 3, 2022, which is specifically incorporated by reference herein.

### BACKGROUND

Severe ankle joint pain is a debilitating affliction to suffering individuals. It causes not only physical degradation, but emotional and psychological degradation. Severe ankle joint pain afflicts a variety of demographics, including but not limited to the elderly, wounded warriors, accident victims, and athletes. Across demographics, individuals may suffer from severe and potentially chronic ankle joint pain due to osteoarthritis (i.e., severe ankle joint pain caused by a substantial reduction or elimination of cartilage within the ankle joint) and/or trauma, for example. This can result in bone-on-bone contact within the joint. For some suffering individuals, the pain has reached a point where they are willing to undergo amputation. An unmet need for these individuals is an effective, non-invasive (i.e., not requiring surgery), and non-permanent (unlike amputation) solution for severe ankle joint pain. Further, even if an invasive and/or permanent solution is later required, a non-invasive solution is useful to delay that requirement.

Some existing non-invasive and non-permanent solutions for their severe ankle joint pain include joint bracing and/or biasing elements, such as arch supports, orthotics, and splints. However, these bracing and/or biasing elements do not address loading applied to the joint, rather they stabilize articulation of the ankle joint. For individuals suffering severe ankle joint pain (e.g., due to osteo-arthritis or injury), merely stabilizing articulation of the ankle joint may be an insufficient solution.

Other existing non-invasive and non-permanent solutions for severe ankle joint pain include offloading braces that rely on a fixed suspension of the user's afflicted ankle joint, thereby offloading it. However, by suspending the ankle joint and replacing its contact with the ground with another device, one of the user's legs is elongated with reference to the other non-injured leg. This can cause additional issues with the user's non-injured leg over time as the user adjusts their gait to compensate. Further, by suspending the ankle joint, any articulation previously offered by the ankle joint in walking is forfeit. Such offloading braces can yield an unnatural “peg-like” quality to the user's gait.

### SUMMARY

Implementations described and claimed herein address the foregoing problems by providing an ankle joint distraction device comprising a calf cuff including a calf tightening mechanism to selectively secure the calf cuff to a user's calf and a foot cuff including a foot tightening mechanism to selectively secure the user's foot to a footplate. The footplate underlies the user's foot.

A distraction assembly has a first distal end pivotally connected to the foot cuff and a second distal end fixedly connected to the calf cuff. The distraction assembly is positioned between the foot cuff and the calf cuff. The distraction assembly distracts the user's ankle joint, the distraction force varying based on the user's state of activity but maintaining the user's foot in contact with the footplate. The distraction force pulls the user's foot from the footplate and therefore the ground absent action from the foot cuff. The distraction force overcomes the user's body weight, and dynamic forces experienced with the user's gait. Hence, the distraction force can be adjusted to accommodate load varying based on the user's state of activity to ensure the user's foot remains in contact with but unloaded from the footplate.

The calf cuff, distraction assembly, and the foot cuff may work in tandem to create distraction in the ankle joint through dual-action functionality. The calf cuff is tightened to the user's calf, followed by actuating the distraction assembly, then the foot cuff is tightened around the foot. In so doing, the foot cuff acts to pull the foot away from the calf and toward the footplate, thereby creating a joint spacing referred to herein as distraction. The distraction assembly pushes the calf upward away from the foot, thereby creating distraction and redirecting body weight and dynamic forces away from the ankle joint.

Other implementations are also described and recited herein.

### BRIEF DESCRIPTIONS OF THE DRAWINGS

FIG. 1 is a perspective view of an example adjustable ankle joint distraction device according to the presently disclosed technology attached to a user's leg.

FIG. 2 is a perspective view of an example calf cuff for an adjustable ankle joint distraction device according to the presently disclosed technology.

FIG. 3 is a side view of an example foot cuff for an adjustable ankle joint distraction device according to the presently disclosed technology.

FIG. 4 is a perspective view of an example distraction assembly for an adjustable ankle joint distraction device according to the presently disclosed technology.

FIG. 5 is a perspective view of an example linear ratchet mechanism according to the presently disclosed technology.

FIG. 6 illustrates an example system diagram of a computing system used to control an adjustable ankle joint distraction device according to the presently disclosed technology.

FIG. 7 illustrates example operations for using an adjustable ankle joint distraction device according to the presently disclosed technology.

### DETAILED DESCRIPTIONS

The presently disclosed adjustable ankle joint distraction devices offload some or a majority of the load on a user's ankle joint, while preserving contact of the user's foot with the ground and articulation of the user's ankle joint with reference to the ground. While much of the following disclosure is directed to use of an ankle joint offloading or distraction device when the user is resting or walking, the ankle joint distraction device may be used during a variety of resting states (e.g., lying prone and sitting) and active and/or dynamic states (e.g., standing, walking, running, and jumping). The following distraction devices may also be applicable to address high-ankle sprains and other injuries when offloading or stabilizing a joint is desired. The following distraction devices may also be used as a pre-operation/post-operation walking shoe or boot or a drop foot support.

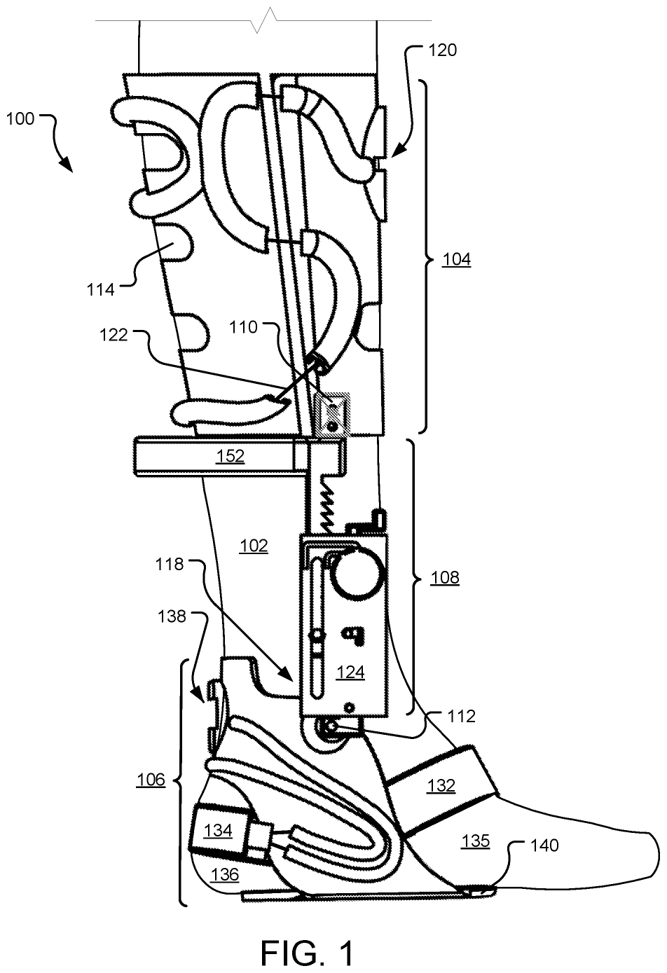

FIG. 1 is a perspective view of an example adjustable ankle joint distraction device (or offloading device) 100 according to the presently disclosed technology attached to a user's leg 102. The distraction device 100 includes a calf cuff 104 connected to a foot cuff 106 with a distraction assembly 108 positioned therebetween. The distraction assembly 108 is mechanically connected to the calf cuff 104 at fixed attachment 110 and to the foot cuff 106 a pivoting attachment 112. The distraction assembly 108, as well as the calf cuff 104 and the foot cuff 106, are all adjustable so that a desired distraction force is achieved by the distraction device 100.

In various implementations, a force of approximately 35 lbs. (or 20-50 lbs.), which is referred to herein as a resting distraction force, is sufficient to create space in the ankle joint and thereby significantly reduce ankle joint pain in a resting state. However, as the distraction device 100 is designed to be used while walking, the distraction force of approximately 20 lbs. is added to a user's weight to calculate the total weight that the distraction device 100 supports (referred to herein as an active distraction force). A dynamic distraction force may be added to the active distraction force whenever the user's heel strikes the ground during activity (e.g., during walking, this load can be reflected as a 1.6 multiplier to the active distraction load (referred to herein as dynamic distraction or offloading force)). Thus, the calf cuff 104 is specifically designed to remain in place on the user's calf 114, which is under a significant active distraction force. For example, for a 150 lb. user, the calf cuff 104 may support a dynamic distraction force of approximately 300 lbs. Further, the distraction force is not constant on the distraction device 100, rather it varies periodically from the resting distraction force to the active distraction force, in some cases in an approximately sinusoidal mode (e.g., while the user is walking).

The calf cuff 104 can transfer the resting, active and/or dynamic distraction force from the distraction device 100 to the user's calf 114 without injuring the user's soft calf tissue or obstructing the user's vascular and/or neural system within their calf 114, while maintaining an adequate level of comfort for the user. Accordingly, the calf cuff 104 has a relatively large surface area to distribute the distraction force across a large portion of the user's calf 114. For example, the calf cuff 104 entirely circumscribes the user's calf starting just below the user's knee (not shown) and extending a substantial distance down the user's lower leg (i.e., a portion of the user's leg 102 that lies between the user's knee and the user's ankle 118). In some implementations, the calf cuff 104 covers at least 25% of the user's lower leg centered on the user's calf 114.

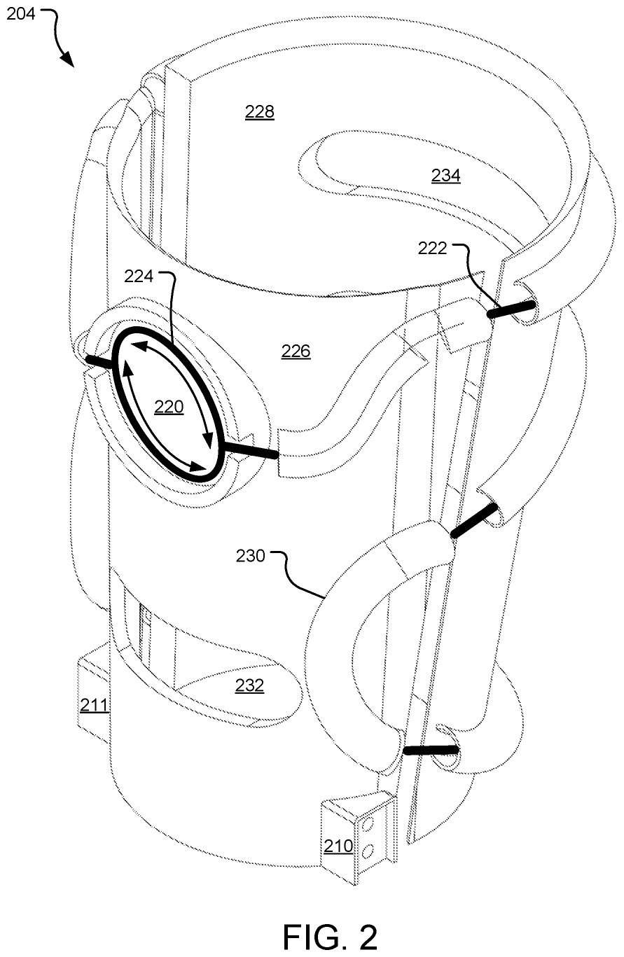

The calf cuff 104 includes a tightening mechanism 120, which provides a uniform constriction force distributed across the calf cuff 104 to adequately secure to the user's calf 114, while minimizing force applied per unit of contact area with the user's calf 114 and staying in position under the resting, active, and/or dynamic distraction force. The tightening mechanism 120 includes a continuous loop of cable 122 that wraps around the calf cuff 104 and terminates at a ratcheting adjuster 124 that allows the user to selectively tighten or loosen the calf cuff 104. Further details regarding the tightening mechanism 120 are discussed below with reference to FIG. 2.

In various other implementations, the tightening mechanism 120 may include laces, ratcheting belts, clamps, pneumatic tubes, etc. Further, the user's calf 114 may include no hair, some hair, or a lot of hair and will perspire to a varying degree depending on the user and their activity level. The calf cuff 104 may be made of a breathable material (e.g., one or more layers of fabric), with or without supplementary holes to facilitate breathability, that can grip skin with differing amounts of hair. The breathable material of the calf cuff 104 may also be anti-microbial and/or deodorizing to reduce or prevent offending odors caused by the tight relation between the user's calf 114 and the calf cuff 104 and potentially lengthy durations that the user may wear the calf cuff 104.

The foot cuff 106 may have similar design constraints to the calf cuff 104, with some distinctions. The foot cuff 106 is designed to remain in place on the user's foot 135, specifically around the user's hindfoot and/or midfoot. In various implementations, the foot cuff 106 may circumscribe the user's hindfoot by extending over the ankle 118 and beneath heel 136. In various implementations, the foot cuff 106 may also extend partially or fully into the user's midfoot. For example, the foot cuff 106 may include a bridge strap 132 that extends over the top of the foot 135 above the midfoot and a heel strap 134 that extends behind the foot 135 above the ankle 118 that connects with a portion of the foot cuff 106 that extends beneath the heel 136 to fully circumscribe the foot 135. In other implementations, the foot cuff 106 may avoid extension into some or all of the midfoot and forefoot to avoid restraining the user's metatarsal bones, which are to remain free and not locked in place. The foot cuff 106 is under an equal and opposite distraction force as described above with reference to the calf cuff 104, with a difference being that the in-active distraction force is subtractive rather than additive to the active distraction force when measuring total load on the foot cuff 106.

The foot cuff 106 transfers the distraction force from the distraction assembly 108 to the footplate 140, thereby bypassing the ankle 118 without injuring the user's ankle tissue or obstructing the user's vascular and/or nervous systems within their ankle 118, while maintaining an adequate level of comfort at the ankle 118. The cuff 106 transfers some or all of the active distraction force from the ground, through the footplate 140, to the distraction assembly 108, while eliminating or minimizing load applied to the user's ankle 118.

The foot cuff 106 may include a tightening mechanism 138 that provides downward force to the heel strap 134 and a forefoot or bridge strap 132 that provides downward force to the entire foot 135. The tightening mechanism 138 may operate in one of two example modalities. First, the calf cuff 104 is actuated first, while the tightening mechanism 138 on the foot cuff 106 remains open or fully extended. Once the calf cuff 104 is actuated (e.g., compressed to remove all the slack or movement in the calf soft tissue), the tightening mechanism 138 is actuated to pulls the heel strap 134 and forefoot strap 132 downward across the heel 136 and foot 135 to effectively pull the entire foot 135 away for the calf 114, which in turns creates a desired distraction or distraction force to expand the ankle joint (e.g., approximately 20-31bs). Second, the foot cuff 106 tightening mechanism 138 is actuated before the distraction assembly 108 is actuated. The tightening mechanism 138 applies a uniform downward force to the heel strap 134 and a forefoot or bridge strap 132 to adequately secure the foot 135 to the footplate 140. This force may be slightly greater than the distraction force applied by the distraction assembly 108.

In various implementations, the tightening mechanism 138 may be focused on tightening the heel strap 134 and/or the forefoot or bridge strap 132, or provide a uniform constriction force across the entire foot cuff 106 (including the heel strap 134 and the forefoot or bridge strap 132) to adequately secure the foot cuff 106 to the foot 135 and stay in position under the distraction force. The tightening mechanism 138 may be similar to that described above with reference to the tightening mechanism 120 for the calf cuff 104. The foot cuff 106 prevents a gap from forming between footplate 140 and the foot 135, even when the distraction force applied by the distraction assembly 108 is at a maximum value. This is distinct from various prior art solutions where a gap is permitted to be created, and sometimes is desired. The absence of such a gap in the presently disclosed technology is advantageous in that a gap elongates the user's injured leg with reference to the user's other leg, which can cause additional injury over time as the user adjusts their gait to compensate. Further, by suspending the user's foot above the footplate 140, the user loses the sense of contact with the ground and can yield an unnatural “peg-like” quality to the user's gait. The foot cuff 106 may also incorporate a breathable material (e.g., a breathable liner for all or discrete areas of the foot cuff 106), with or without supplementary holes in the foot cuff 106 to facilitate breathability. The foot cuff 106 may further incorporate anti-microbial and/or deodorizing materials.

The distraction assembly 108 is a rigid member that is adjustable in working length so that a desired distraction force is achieved by the distraction assembly 108 by pushing the calf cuff 104 and the foot cuff 106 apart. The distraction assembly 108 includes a ratcheting mechanism and a lifting handle 152 that together function as a macro-adjuster that a user may utilize to extend the distraction assembly 108, referred to herein as making macro-adjustments to the distraction assembly 108. Once the user has extended the distraction assembly 108 close to the desired distraction magnitude, the user can utilize a micro-adjuster to make micro-adjustments to the length of the distraction assembly 108 to achieve the desired distraction magnitude.

In various other implementations, the distraction assembly 108 is that of a piston and cylinder arrangement, with pneumatic pressure, hydraulic pressure, and/or a mechanical screw used to adjust the resting working length of the distraction assembly 108, which in turn and as a result adjusts the distraction magnitude provided by the distraction assembly 108. The distraction assembly 108 may also include a spring mechanism and/or a damper mechanism to cushion changing forces applied on the distraction device 100 when the user is in an active state. However, the rigid distraction assembly 108 may be desirable over a compressible distraction assembly, such as a shock absorber or strut, in that the rigid distraction assembly 108 maintains distraction in the user's ankle 118 without generating a gap between the foot 135 and the footplate 140 for the foot cuff 106. Such a gap is undesirable, as discussed above.

The fixed attachment 110 and the pivoting attachment 112 serve to connect opposing ends of the distraction assembly 108 to the calf cuff 104 and the foot cuff 106, respectively. These attachments 110, 112 carry distraction force applied by the distraction assembly 108 to the calf cuff 104 and the foot cuff 106, and carry a load from the ground, through the foot cuff 106, the distraction assembly 108, and the calf cuff 1, and ultimately to the user's calf 114. While depicted as screwed or bolted connections in FIG. 1, the attachments 110, 112 may be of any format, size, and shape adequate to connect the opposing ends of the distraction assembly 108 to the calf cuff 104 and the foot cuff 106 and provide sufficient spacing of the distraction assembly 108 from the user's lower leg.

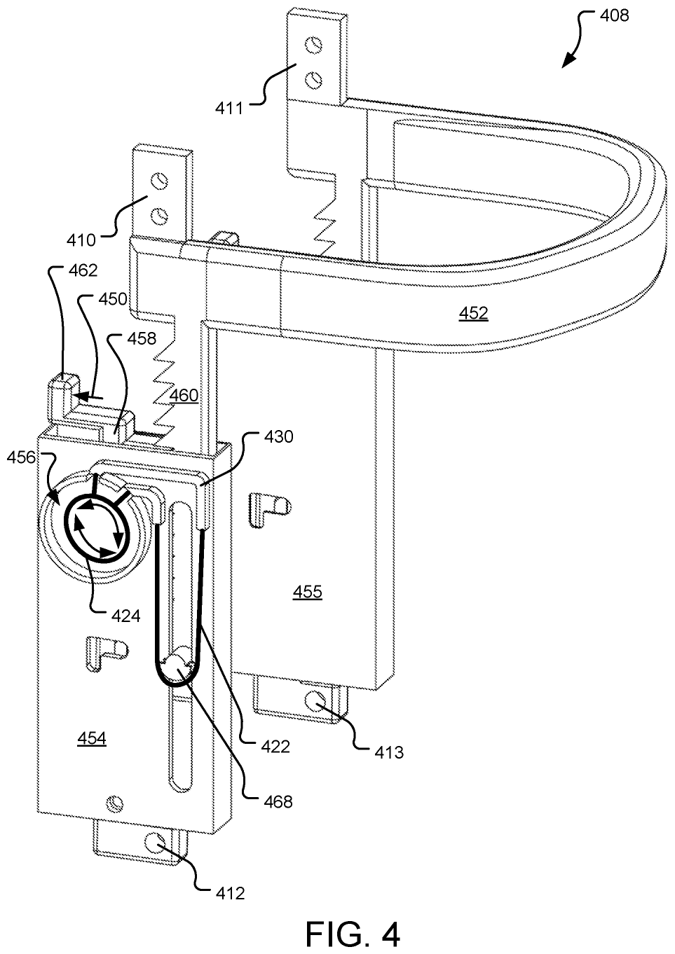

In some implementations, the distraction assembly 108 may include a pair of ratcheting mechanisms (not shown, see e.g., ratcheting mechanisms 454, 455 of FIG. 4 and associated attachments on opposite sides of the user's leg 102 to balance the distraction force applied by the distraction assembly 108. This avoids unwanted torsional forces that occur due to an offset that a singular ratcheting mechanism may have from the user's leg 102. In implementations where a singular ratcheting mechanism is used, the spacing of the distraction assembly 108 from the user's leg 102 may be kept to a minimum to minimize the unwanted torsional forces. In other implementations, the distraction assembly 108 may be replaced with a different type of distraction device, such as one including threaded rods with cam buckles, a system of pulleys and cables, and/or a ratcheting or otherwise selectively extending mechanical structure.

The distraction device 100 may further include a shock absorbing pad (not shown) between the footplate 140 and the heel 136 to cushion the foot 135 from varying forces, and decreasing peak load, particularly when the user is in an active state. In various implementations, the shock absorbing pad 166 may be polymeric or elastomeric, for example. The shock absorbing pad may further be made of foam, gel, or rubber, for example. In some implementations, the shock absorbing pad 166 is designed to mimic the load response characteristics of human cartilage to maximize effectiveness of distraction device 100 and potentially provide a more natural response to the user. Further, the shock absorbing pad 166 may be designed in conjunction with the remaining components of the distraction device 100 to mimic the response characteristics of an uninjured human lower leg, further potentially providing a more natural response to the user.

Various components of the distraction device 100 may be constructed of molded plastics, carbon fiber, and/or fiberglass, with metal reinforcement where required (e.g., at the attachments 110, 112).

FIG. 2 is a perspective view of an example calf cuff 204 for an adjustable ankle joint distraction device (or offloading device) according to the presently disclosed technology. The calf cuff 204 is connected to a foot cuff (not shown, see e.g., foot cuff 106 of FIG. 1) with one or more distraction assemblies (not shown, see e.g., distraction assembly 108 of FIG. 1) positioned therebetween to create the distraction device. Here, distracting assemblies extending from each side of the calf cuff 204 are mechanically connected to the calf cuff 204 at fixed attachments 210, 211 (e.g., bolted or screwed connections to the calf cuff 204). The calf cuff 204 is specifically designed to remain in place on a user's calf (not shown see e.g., calf 114 of FIG. 1), which is under a significant resting, active, and/or dynamic force that changes in magnitude over time, particularly as the user walks.

The calf cuff 204 can transfer the resting, active, and/or dynamic distracting force from the distracting device to the user's calf without injuring the user's soft calf tissue or obstructing the user's vascular and/or nervous systems within their calf, while maintaining an adequate level of comfort for the user. Accordingly, the calf cuff 204 has a relatively large surface area to distribute the distracting force across a large portion of the user's calf. Specifically, the calf cuff 204 includes two rigid cuff sections 226, 228 that are sized and shaped to entirely or substantially (greater than 90%) circumscribe the user's calf starting just below the user's knee and extending a substantial distance down the user's lower leg. The rigid cuff sections 226, 228 distribute the compression force across the user's soft calf tissue without pressure points that may be created in a calf cuff with a smaller surface area or flexible body that may deform under the compression force. While the calf cuff 204 has two rigid cuff sections 226, 228, other implementations may have greater or fewer rigid cuff sections. In some implementations, the rigid cuff sections 226, 228 are custom molded (or semi-custom thermoformed) to the user's lower legs. This ensures that an interior surface contour of the rigid cuff sections 226, 228 closely matches the contour of the user's lower legs, which further reduces the risk of creating pressure points.

The calf cuff 204 includes a tightening mechanism 220, which provides a uniform constriction force distributed across the calf cuff 204 to adequately secure to the user's calf, while minimizing force applied per unit of contact area with the user's calf and staying in position under the resting, active, and/or dynamic distracting force. The tightening mechanism 220 includes a continuous loop of cable 222 that wraps around the calf cuff 204 and terminates at a ratcheting adjuster 224 that allows the user to selectively tighten or loosen the calf cuff 204. The cable 222 extends through a series of guide tubes (e.g., guide tube 230) that define a fixed path for the cable 222 to wrap around the calf cuff 204. This ensures that the cable 222 remains distributed across the length of the calf cuff 204 and evenly applies pressure to the calf cuff 204 when the tightening mechanism 220 is tightened. The ratcheting adjuster 224 may include a mechanical advantage (or force multiplication) mechanism so that the user may sufficiently tighten the calf cuff 204 even if the user has limited hand strength. In some implementations, the tightening mechanism 220 is a BOA® Fit System. In other implementations, the tightening mechanism 220 may include laces, ratcheting belts, clamps, pneumatic tubes, etc. in addition to or in lieu of that shown in FIG. 2.

The calf cuff 204 may include a breathable liner (e.g., one or more layers of fabric), with or without supplementary holes 232, 234 though the rigid cuff sections 226, 228 to facilitate breathability. The calf cuff 204 may also include anti-microbial and/or deodorizing materials to reduce or prevent offending odors caused by the tight relation between the user's calf and the calf cuff 204 and potentially lengthy durations that the user may wear the calf cuff 204. Further, the tightening mechanism 220 may include a load limiter to prevent overtightening and/or a manual release as a failsafe.

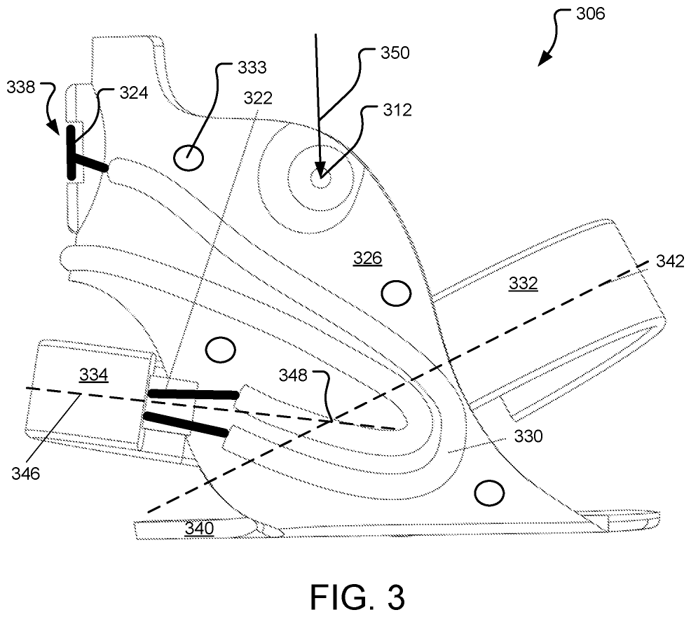

FIG. 3 is a side view of an example foot cuff 306 for an adjustable ankle joint distraction device (or offloading device) according to the presently disclosed technology. The foot cuff 306 is connected to a calf cuff (not shown, see e.g., calf cuff 104 of FIG. 1) with one or more distraction assemblies (not shown, see e.g., distraction assembly 108 of FIG. 1) positioned therebetween to create the distraction device. Here, distraction assemblies extending from each side of the foot cuff 306 are mechanically connected to the foot cuff 306 at pivoting attachments (e.g., pivoting attachment 312, such as a bolted or screwed connections to the foot cuff 306). The foot cuff 306 is specifically designed to remain in place on a user's foot (not shown see e.g., foot 135 of FIG. 1), which is under a significant resting, active, and/or dynamic distraction force that changes in magnitude over time, particularly as the user walks.

The foot cuff 306 can transfer the resting, active, and/or dynamic distraction force from the distraction device to the user's foot without injuring the user's soft foot tissue or obstructing the user's vascular and/or nervous systems within their foot, while maintaining an adequate level of comfort for the user. The foot cuff 306 may transfer some or all forces to the ground while bypassing the ankle joint. Accordingly, the foot cuff 306 has a relatively large surface area to distribute the distraction force across a large portion of the user's foot. Specifically, the foot cuff 306 includes a rigid cuff section 326 that are sized and shaped to cover a majority of the sides of the user's hindfoot and/or midfoot and connect across the rear of the user's hindfoot above the user's heel. The foot cuff 306 further includes a footplate 340 that is connected to each side of the rigid cuff section 326 and underlies a majority of the underside of the user's hindfoot and/or midfoot.

The rigid cuff section 326 serves to carry the distraction force from the distraction assembly to the foot plate 340. In some implementations, the rigid cuff section 326 further distributes compression force across the user's hindfoot and/or midfoot without pressure points that may be created in a foot cuff with a smaller surface area or flexible body that may deform under the compression force. While the foot cuff 306 has a singular rigid cuff section 326, other implementations may have more rigid cuff sections linked together. In some implementations, the rigid cuff section 326 is custom molded (or semi-custom thermoformed) to the user's feet. This ensures that an interior surface contour of the rigid cuff section 326 closely matches the contour of the user's feet, which further reduces the risk of creating pressure points.

The foot cuff 306 further includes a bridge strap 332 that extends over the top of the foot above or in front of the ankle that connects with the rigid cuff section 326 on opposing sides. This allows the foot cuff 306 to fully circumscribe the foot and secure the foot to the foot cuff 306 along bridge strap axis 342. The foot cuff 306 still further includes a heel strap 334 that extends around the rear of the foot at or above the heel that also connects with the rigid cuff section 326 on opposing sides. This allows the foot cuff 306 to fully circumscribe the foot and secure the foot to the foot cuff 306 along heel strap axis 346. As the strap axes 342, 346 intersect at point 348, and the distraction assembly applies a downward-oriented force (illustrated by arrow 350 at the pivoting attachment 312, the foot is fully constrained by the foot cuff 306 in anterior/posterior directions, as well as up/down directions. The relative angles of the strap axes 342, 346 are illustrated as examples only, implementations of the foot cuff 306 applied to a user's foot may adopt different angles. The foot is further fully constrained in the medial/lateral directions by the rigid cuff section 326 extending up both sides of the foot, as discussed above. As a result, the foot is secured within the foot cuff 306 in all directions.

The foot cuff 306 includes a tightening mechanism 338, which provides tightening force to one or both of the bridge strap 332 and the heel strap 334. The tightening mechanism 338 may further provide a uniform constriction force distributed across the foot cuff 306 to adequately secure to the user's foot, while minimizing force applied per unit of contact area with the user's foot and staying in position under the resting, active, and/or dynamic distraction force.

The tightening mechanism 338 includes a continuous loop of cable 322 that wraps around the foot cuff 306 and terminates at a ratcheting adjuster 324 that allows the user to selectively tighten or loosen one or both of the bridge strap 332 and the heel strap 334. The cable 322 extends through a series of guide tubes (e.g., guide tube 330) that define a fixed path for the cable 322 to wrap around the foot cuff 306. This ensures that the cable 322 remains distributed across the length of the foot cuff 306 on each side and evenly applies downward pulling force to both the bridge strap 332 and the heel strap 334 simultaneously (and/or pressure to the foot cuff 306) overall when the tightening mechanism 338 is tightened.

The cable 322 further extends through the heel strap 334 (and in some implementations, the bridge strap 332) to incorporate one or both straps 332, 334 into the tightening mechanism 338. The ratcheting adjuster 324 may include a mechanical advantage (or force multiplication) mechanism so that the user may sufficiently tighten the foot cuff 306 even if the user has limited hand strength. In some implementations, the tightening mechanism 338 is a BOA® Fit System. In other implementations, the tightening mechanism 338 may include laces, ratcheting belts, clamps, pneumatic tubes, etc. in addition to or in lieu of that shown in FIG. 3.

The tightening mechanism 338 and straps 332, 334 are set such that the user's foot maintains contact with the footplate 340, but the ankle joint is substantially unloaded. This prevents a gap from forming between footplate 340 and the foot, even when the distraction force applied by the distraction assembly is at a maximum value. This is distinct from various prior art solutions where a gap is permitted to be created, and sometimes is desired. The absence of such a gap in the presently disclosed technology is advantageous in that a gap elongates the user's injured leg with reference to the user's other leg, which can cause additional injury over time as the user adjusts their gait to compensate. Further, by suspending the user's foot above the footplate 340, the user loses the sense of contact with the ground and can yield an unnatural “peg-like” quality to the user's gait.

In various implementations, the foot cuff 306 may be useable with any style of shoe, or without a shoe at all. Further, the foot cuff 306 may be fitted inside or outside of a shoe. The foot cuff 306 may include a breathable liner (e.g., one or more layers of fabric), with or without supplementary holes (e.g., hole 333) though the rigid cuff section 326 to facilitate breathability. The foot cuff 306 may also include anti-microbial and/or deodorizing materials to reduce or prevent offending odors caused by the tight relation between the user's foot and the foot cuff 306 and potentially lengthy durations that the user may wear the foot cuff 306. Further, the tightening mechanism 338 may include a load limiter to prevent overtightening and/or a manual release as a failsafe.

One or both of the calf cuff 204 of FIG. 2 and the foot cuff 306 of FIG. 3 may include various sensors to monitor the calf cuff 204 in relation to the user's calf and/or the foot cuff 306 in relation to the user's foot. For example, the calf cuff 204 and/or foot cuff 306 may include one or more of: moisture sensor(s) to detect moisture build-up between the calf cuff 204/calf and/or foot cuff 306/foot, fan(s) (perhaps selectively powered) connected to the moisture sensor(s) to ensure that the calf cuff 204/foot cuff 306 are adequately ventilated, strain gauge(s) or pressure sensor(s) to monitor the calf/foot tightening mechanisms 220, 338 to ensure that pressure applied to the calf/foot is within an acceptable range that balances distraction device effectiveness and calf/foot health and comfort, blood flow or pulse sensor(s) to monitor calf/foot health and comfort.

FIG. 4 is a perspective view of an example distraction assembly 408 for an adjustable ankle joint distraction device (or offloading device) according to the presently disclosed technology. The distraction assembly 408 connects a foot cuff (not shown, see e.g., foot cuff 106 of FIG. 1) to a calf cuff (not shown, see e.g., calf cuff 104 of FIG. 1) to create the distraction device. The distraction assembly 408 extends between opposing sides of the foot cuff and the calf cuff and is mechanically connected to the foot cuff at pivoting attachments 412, 413 (e.g., bolted or screwed connections to the foot cuff) and to the calf cuff at fixed attachments 410, 411 (e.g., bolted or screwed connections to the calf cuff).

The distraction assembly 408 is a rigid member that is adjustable in working length so that a desired distraction magnitude is achieved by the distraction assembly 408 by pushing the calf cuff and the foot cuff apart. This is technically advantageous over a compressible member, such as a shock absorber or strut in that the rigid distraction assembly 408 (working in conjunction with the calf cuff and the foot cuff) maintains distraction in the user's ankle joint without generating a gap between the user's foot and a footplate for the foot cuff. Such a gap is undesirable, as discussed above. In some implementations, however, the distraction assembly 408 can include a spring mechanism to return some amount of impact force with the ground to the user's gait as the user walks.

The distraction assembly 408 includes a pair of ratcheting mechanisms 454, 455 and a lifting handle 452 that together function as a macro-adjuster that a user may utilize to extend the distraction assembly 408, referred to herein as making macro-adjustments to the distraction assembly 408. The dual ratcheting mechanisms 454, 455 are generally aligned parallel with and equidistant from the user's ankle joint, thereby preventing any unnecessary and undesirable torque or other forces on the user's ankle joint. By pulling upward on the lifting handle 452, the user extends the distraction assembly 408 and attached calf cuff with reference to the foot cuff. The dual ratcheting mechanisms 454, 455 have sufficient working length to first take up any slack in the distraction assembly 408, then any slack in the user's skin and soft tissue, and finally apply a desired distraction force to the user's ankle joint. In other implementations, the lifting handle 452 is omitted and replaced with a pair of smaller lifting handles positioned on opposing sides of the distraction assembly 408. Further, other distraction assemblies may include singular lifting handle connected to a singular ratcheting mechanism, in some implementations positioned at a rear of the distraction assembly behind the user's leg.

The ratcheting mechanisms 454, 455 each include matching toothed racks (see e.g., toothed racks 458, 460 of ratcheting mechanisms 454, 455). The toothed rack 460 is contiguous with the lifting handle 452, while the toothed rack 458 is contained within the body of the ratcheting mechanism 454 and is spring-loaded against the teeth of the toothed rack 460. A similar configuration is found within the ratcheting mechanism 455. This configuration allows the ratcheting mechanisms 454, 455 together to function as a macro-adjuster to extend stepwise one tooth at a time and maintain that position when the lifting handle 452 is released.

Once the user has extended the distraction assembly 408 close to the desired distraction magnitude, the user can utilize a micro-adjuster 456 to make micro-adjustments to the length of the distraction assembly 408 to achieve the desired distraction magnitude. Specifically, the micro-adjuster 456 includes a continuous loop of cable 422 that wraps around lifting hook 468 and terminates at a ratcheting adjuster 424 that allows the user to selectively raise or lower the lifting hook 468 and thereby extend or retract the distraction assembly 408, respectively. The cable 422 extends through a pair of guide tubes (e.g., guide tube 430) that define a fixed path for the cable 422 to extend from the ratcheting adjuster 424 to the lifting hook 468. The micro-adjuster 456 permits micro-adjustments to the length of the distraction assembly 408 between the stepwise one tooth at a time macro-adjustments of the matching toothed racks of the ratcheting mechanisms 454, 455. In many implementations, it is important for the user to be able to make micro-adjustments to the length of the distraction assembly 408 throughout the day so that the distraction assembly 408 remains effective and relatively comfortable. In some implementations, the micro-adjuster 456 is a BOA® Fit System. Further, the micro-adjuster 456 may utilize force multiplication to decrease the user force required to adjust the length of the distraction assembly 408. Still further, the user may make marks on the ratcheting mechanisms 454, 455 to visually indicate preferred positions of the lifting hook 468 to achieve the desired distraction magnitude.

The toothed racks contained within the bodies of their respective ratcheting mechanisms (e.g., toothed rack 458 within ratcheting mechanism 454) each include a release tab (e.g., release tab 462). When the release tab is pressed in a direction that separates the toothed racks within a ratcheting mechanism (e.g., in the direction of arrow 450), the teeth of the toothed racks disengage, and the ratcheting mechanism is released.

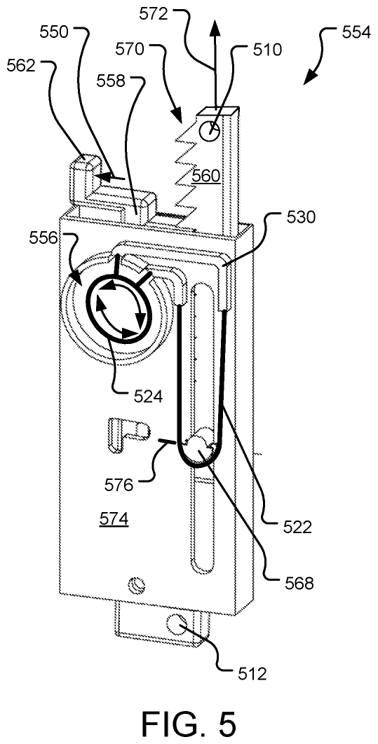

FIG. 5 is a perspective view of an example linear ratchet mechanism 554 according to the presently disclosed technology. In some implementations, the linear ratchet mechanism 554 may be used for an adjustable ankle joint distraction device (or distraction or distraction device) according to the presently disclosed technology, as shown in FIG. 4 and described above. In other implementations, the linear ratchet mechanism 554 may be used for creating joint distraction in any injured joint in the human body. In still further implementations, the linear ratchet mechanism 554 may be used in any application where a linear separation ratcheting mechanism is desired that can quickly make macro-adjustments and also micro-adjustments, all in a compact and lightweight package.

The linear ratchet mechanism 554 is a rigid member that is adjustable in working length to function as a macro-adjuster so that a desired length between attachment points 510, 512 is achieved. Macro-adjustments to the linear ratchet mechanism 554 are made by generally pulling the attachment points 510, 512 apart using an extension force to selectively create extension of the linear ratchet mechanism 554. The linear ratchet mechanism 554 includes a moveable linear rack 560 with teeth 570 including the first attachment point 510 at its distal end. A spring-loaded pawl 558 engages the teeth 570 and permits movement of the linear rack 560 in a singular linear direction illustrated by arrow 572 with reference to a mechanism housing 574 for the linear ratchet mechanism 554. The spring-loaded pawl 558 may include a toothed rack (not shown) matching that of the linear rack 560, though other configurations of the spring-loaded pawl 558 are contemplated herein. The mechanism housing 574 encloses much of the spring-loaded pawl 558 and a proximal end of the moveable linear rack 560. The moveable linear rack 560, including the first attachment point 510 extends out of a first end of the mechanism housing 574. A second end of the mechanism housing 574 includes the second attachment point 512 fixed thereto. This configuration allows the linear ratchet mechanism 554 to extend stepwise one tooth at a time and maintain that position when the extension force is released.

Because the linear ratchet mechanism 554 can only stop backward motion (i.e., motion opposite that of the arrow 572) at discrete points (i.e., at tooth boundaries), the linear ratchet mechanism 554 naturally permits a limited amount of backward motion. This backward motion, which is limited to a maximum distance equal to the spacing between the teeth 570, is referred to herein as backlash. To address the backlash, the linear ratchet mechanism 554 may include a ratcheting micro-adjuster 556 that drives movement of the moveable linear rack 560 in the singular linear direction and locks the moveable linear rack 560 in position within its backlash between tooth boundaries. In use, once a user has extended the linear ratchet mechanism 554 close to a desired length, the user can utilize the ratcheting micro-adjuster 556 to make micro-adjustments to the length of the linear ratchet mechanism 554 to achieve the desired length.

Specifically, the micro-adjuster 556 includes a continuous loop of cable 522 that wraps around lifting hook 568, both distal ends of which terminate at a ratcheting adjuster 524, which may include a rotating handle. Rotation of the ratcheting adjuster 524 allows the user to selectively raise or lower the lifting hook 568 by shortening or elongating the continuous loop of cable 522, and thereby extending or retracting the linear ratchet mechanism 554 within backlash between tooth boundaries. The micro-adjuster 556 may provide mechanical assistance (e.g., force multiplication) of a user's input to extend the linear ratchet mechanism 554. The cable 522 extends through a pair of guide tubes (e.g., guide tube 530) that define a fixed path for the cable 522 to extend from the ratcheting adjuster 524 to the lifting hook 568. The micro-adjuster 556 permits micro-adjustments to the length of the linear ratchet mechanism 554 between the stepwise one tooth at a time macro-adjustments of the moveable linear rack 560. In joint distraction or offloading implementations, it is important for the user to be able to make micro-adjustments to the length of the linear ratchet mechanism 554 throughout the day so that a corresponding distraction device remains effective and relatively comfortable. In some implementations, the micro-adjuster 556 is a BOA® Fit System. Further, the micro-adjuster 556 may utilize force multiplication to decrease the user force required to adjust the length of the linear ratchet mechanism 554.

The spring-loaded pawl 558 includes a release tab 562 extending outside of the mechanism housing 574. When the release tab 562 is pressed in a direction that separates the spring-loaded pawl 558 from the teeth 570 of the moveable linear rack 560 (e.g., in the direction of arrow 550), spring force is overcome and the spring-loaded pawl 558 disengages from the teeth 570 of the moveable linear rack 560, thereby releasing the ratcheting mechanism. This permits movement of the moveable linear rack 560 in a direction opposite of the singular linear direction.

In various implementations, the mechanism housing 574 includes a user-selectable preset for returning the first attachment point 510 to a prior position with reference to the second attachment point 512. For example, the user may make a mark 576 on the mechanism housing 574 to visually indicate a preferred position of the lifting hook 568 to quickly return to a desired length of the linear ratchet mechanism 554. The linear ratchet mechanism 554 is further a tool-less design that may be manually adjusted by a user using only their hands. The linear ratchet mechanism 554 may further support greater than 300 lbs.

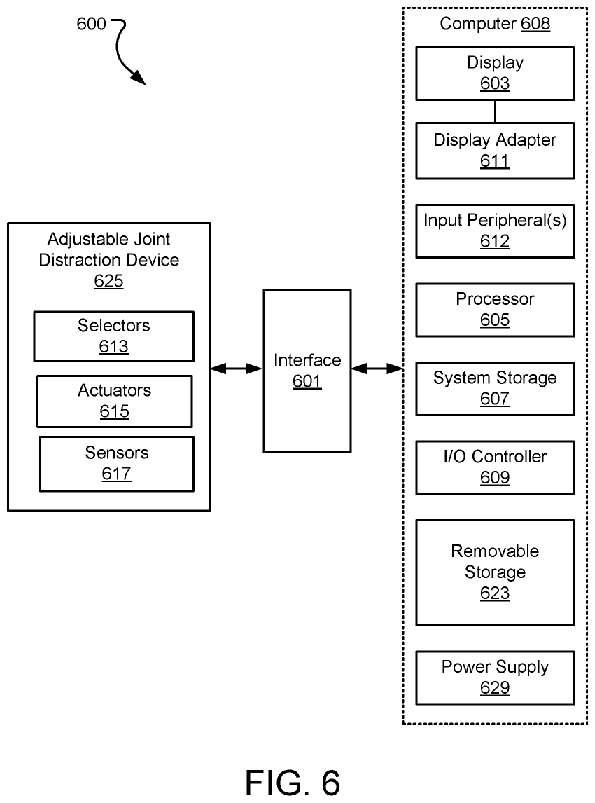

FIG. 6 illustrates an example system diagram of a computing system 600 used to control an adjustable ankle joint distraction device 625 according to the presently disclosed technology. The device 625 may include a variety of selectors 613, actuators 615, and sensors 617, each of which may be controlled and/or monitored by a computer (or microcontroller) 608. While the computing system 600 is illustrated as a singular computer 608 connected to the device 625 via interface 601, some aspects of the computing system 600 may be included within the device 625, while other aspects of the computing system 600 may be selectively connected to the device 625. Further, multiple computers, such as computer 608, may be connected to the device 625.

The sensors 617 are used to monitor the state of the device 625 and its interaction with a user. For example, a distraction assembly (see e.g., distraction assembly 108 of FIG. 1) for the device 625 may include a strain gauge to provide feedback on a distraction magnitude applied by the distraction assembly and/or a displacement sensor to measure extension of the distraction assembly. The device 625 may also include an orientation sensor to detect whether the user is currently in a prone position or an upright position and track changes therebetween, which can help determine if the user is undertaking a resting state or an active state. The orientation sensor may also be used to distinguish between active states (e.g., standing vs. walking).

The sensors 617 may further include biometric monitors to ensure the health of the user's leg. Specifically, a pressure sensor may monitor construction force applied by one or both of a calf cuff (see e.g., calf cuff 104 of FIG. 1) and a foot cuff (see e.g., foot cuff 106 of FIG. 1). Further, the sensors 617 may include pulse oximeters, in some implementations, at the user's calf and at the user's foot to monitor blood flow through the user's leg. Constriction of blood flow is minimized and feedback from the pulse oximeters may drive changes in constriction force applied by one or both of the calf cuff and the foot cuff.

In further implementations, the sensors 617 may include one or more pressure sensors on a footplate (see e.g., footplate 140 of FIG. 1) to ensure contact of the user's foot with the footplate (no gap), but minimizing the force applied to the foot, and/or monitoring the user's gait. A displacement sensor may further monitor position of the calf cuff with reference to the user's calf. This data may be used to detect slippage, which indicates that the calf cuff is insufficiently secured to the user's calf. Moisture sensors on one or both of the calf cuff and the foot cuff may be used to detect skin health and ensure that the user's skin receives adequate ventilation. Temperature sensors may also be included to detect hot spots that might indicate undesirable pressure points or friction.

In various implementations, tightening mechanisms of the calf cuff and the foot cuff, as well as the distraction assembly are manually controlled by the user. The user adjusts the tightening mechanisms to achieve tight but adequately comfortable connections at the user's calf and foot and a distraction magnitude that best alleviates the user's ankle joint pain. In other implementations, the tightening mechanisms of the calf cuff and the foot cuff, as well as the distraction assembly, are controlled by the actuators (or micro-actuators) 615 and associated software (or micro-controllers). The actuators (or micro-actuators) 615 may be motor controlled and powered by a battery or other power source. The software may have fixed programming to achieve and maintain predetermined pressure applied by the calf cuff and the foot cuff, as well as a distraction magnitude applied by the distraction assembly.

The software running the actuators 615 for the calf cuff, the foot cuff, and/or the distraction assembly may incorporate a feedback mechanism for varying the pressure applied by the calf cuff and the foot cuff and the distraction magnitude applied by the distraction assembly. For example, the pressure applied by the calf cuff and the foot cuff, and the distraction magnitude applied by the distraction assembly, may decrease when the user is in a resting state and increase when the user is in an active state. The software may also scale the pressure applied by the calf cuff and the foot cuff and the distraction magnitude applied by the distraction assembly up and down over time to allow the user to adjust to the changed pressure and/or distraction/offloading magnitude/force.

The software running the actuators 615 for the calf cuff, the foot cuff, and/or the distraction assembly may also incorporate machine learning or adaptive programming based on the user's behavior. For example, the software may learn the user's gait while walking and actively adjust the calf cuff, the foot cuff, and/or the distraction assembly as the user walks, for example applying increased distraction magnitude when loading the injured ankle and decreased distraction magnitude when un-loading the injured ankle. For further example, the software may predict and adapt based on type of resting states or active states that it detects that the user has undertaken previously, is currently undertaking, and predicts that the user will undertake. The software may account for the user's typical behavior over the course of a day, week, and/or month to further predict the user's behavior. For example, the software may expect the user to sleep from 10 pm-6 am and take a nap between 1 pm and 2 pm and adjust accordingly to a lower pressure applied by the calf cuff and the foot cuff and the distraction magnitude applied by the distraction assembly during those time periods. In still further implementations, the tightening mechanisms of the calf cuff and the foot cuff, as well as the distraction assembly are selectively manually controlled by the user, automatically controlled by software, or a combination of automatic/manual control for different features of the device 625.

The selectors 613 (e.g., dials, switches, or buttons) are used by the user to adjust the device 625. For example, the device 625 may include and on/off selector, a manual extend/retract selector for the distraction assembly, a manual compress/de-compress selector for the calf cuff, a manual compress/de-compress selector for the foot cuff, an automatic/manual mode selector, and/or a state of pain selector (e.g., a dial ranging from 0-10 in pain level). The setting of the selectors 613 defines how the device 625 operates. The device 625 may further include one or more visual and/or audio indicators of the state of the selectors 613. Still further, the user may interact with a software application (e.g., a mobile app) in addition to or in lieu of the selectors 613. The software application may also provide feedback to the user on the operation of the device 625. Use of the software application and/or the selectors 613 may aid in the function of machine-learning in optimizing the automatic operation of the device 625, including the user's state of pain, which may not otherwise be detectible by the device 625. Finally, one of the selectors 613 (e.g., the on/off selector) may function as a failsafe if the device 625 malfunctions.

In various implementations, the device 625 can upload collected data on its use to a computing network (wired or wireless), for example, timing and duration of its use, the user's resting and active states during use, settings of an associated calf cuff, foot cuff, and distraction assembly, and measurements provided by the sensors 617 within the device 625. The uploaded data may then be analyzed by the user (e.g., using a mobile computing device, such as a smart phone) and/or their health care provider to evaluate a treatment plan and plan further treatment for the user's afflicted ankle joint. Data analytics or analysis may be applied to analyze past performance of the device 625 and make suggestions to optimize future performance of the device 625.

The computer 608 controls and/or monitors the device 625 via an interface 601 (wired or wireless), which provides one or more data connections between the computer 608 and the device 625. The computer 608 includes major subsystems such as a processor 605, system storage 607 (such as random-access memory (RAM) and read-only memory (ROM)), an input/output (I/O) controller 609, removable storage (such as a memory card) 623, a power supply 629, and external devices such as a display screen 603 via a display adapter 611, and various input peripherals 612 (e.g., a mouse, trackpad, keyboard, touchscreen, joystick, and/or smart card acceptance device). The interface 601 may also be used to connect the computer 608 and/or the device 625 to a local or wide area network (such as the Internet) using any network interface system known to those skilled in the art.

Many other devices or subsystems (not shown) may be connected in a similar manner (e.g., servers, personal computers, tablet computers, smart phones, mobile devices, etc.). Also, it is not necessary for all of the components depicted in FIG. 6 to be present to practice the presently disclosed technology. Furthermore, devices and components thereof may be interconnected in different ways from that shown in FIG. 6. Code (e.g., computer software, including mobile applications (apps) to implement the presently disclosed technology may be operably disposed in the device 625, the system storage 607, and/or the removable storage 623.

The computing system 600 may include a variety of tangible computer-readable storage media (e.g., the system storage 607 and/or the removable storage 623) and intangible computer-readable communication signals. Tangible computer-readable storage can be embodied by any available media that can be accessed by the computing system 600 and includes both volatile and non-volatile storage media, as well as removable and non-removable storage media implemented in any method or technology for storage of information such as computer readable instructions, data structures, program modules, and/or other data. Tangible computer-readable storage media includes, but is not limited to, firmware, RAM, ROM, electrically erasable programmable read-only memory (EEPROM), flash memory or other memory technology, optical disc storage, magnetic cassettes, magnetic tape, magnetic disc storage or other magnetic storage devices, or any other tangible medium which can be used to store the desired information, and which can be accessed by the computing system 600.

Intangible computer-readable communication signals may embody computer readable instructions, data structures, program modules, or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include signals traveling through wired media such as a wired network or direct-wired connection, and wireless media such as acoustic, radio frequency (RF), infrared (IR), and other wireless media. Computer-readable storage media as defined herein specifically excludes intangible computer-readable communications signals.

Some implementations may include an article of manufacture which may comprise a tangible storage medium to store logic. Examples of a storage medium may include one or more types of computer-readable storage media capable of storing electronic data, including volatile memory or non-volatile memory, removable or non-removable memory, erasable or non-erasable memory, writeable or re-writeable memory, and so forth. Examples of the logic may include various software elements, such as software components, programs, applications, computer programs, application programs, system programs, machine programs, operating system software, middleware, firmware, software modules, routines, subroutines, operation segments, methods, procedures, software interfaces, application program interfaces (APIs), instruction sets, computing code, computer code, code segments, computer code segments, words, values, symbols, or any combination thereof. In one implementation, for example, an article of manufacture may store executable computer program instructions that, when executed by a computer, cause the computer to perform methods and/or operations in accordance with the described implementations. The executable computer program instructions may include any suitable type of code, such as source code, compiled code, interpreted code, executable code, static code, dynamic code, and the like. The executable computer program instructions may be implemented according to a predefined computer language, manner or syntax, for instructing a computer to perform a certain operation segment. The instructions may be implemented using any suitable high-level, low-level, object-oriented, visual, compiled and/or interpreted programming language.

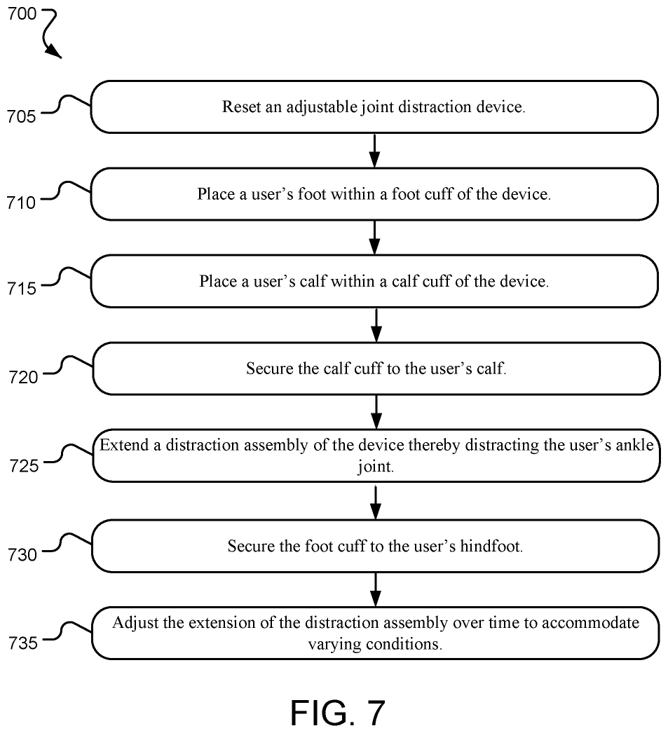

FIG. 7 illustrates example operations 700 for using an adjustable ankle joint distraction device according to the presently disclosed technology. A resetting operation 705 resets the device to a default position. The resetting operation 705 may include fully extending a distraction assembly. The resetting operation 705 may further include resetting tightening mechanisms (e.g., set to a fully extended orientation) for one or both a calf cuff and a foot cuff for the adjustable joint distraction device as well. The distraction assembly may be automatically or manually reset at any point prior to placement on a user's leg.

A first placement operation 710 places a user's foot within a foot cuff of the device. The foot cuff wraps around the user's hindfoot. A second placement operation 715 places a user's calf within a calf cuff of the device. The placement operations 710, 715 may be referred to in combination as the user donning the device.

The calf cuff wraps around the user's calf. A first securing operation 720 secures the calf cuff to the user's calf. The first securing operation 720 may be accomplished using a first tightening mechanism that may be mechanical or electro-mechanical, and manual, partially-automated, or fully-automated, for example.

An extension operation 725 extends the distraction assembly thereby distraction the user's ankle joint. The extension operation 725 may be accomplished in two sub-steps, for example. First, the extension operation 725 is performed by using a macro-adjuster to extend the distraction assembly. The macro-adjuster may be a handle that is used to manually extend the distraction assembly to take up slack in skin and soft tissue. Second, the extension operation 725 is then performed by using a micro-adjuster to further extend the distraction assembly. The micro-adjuster may be used by first orienting the device so that it is unloaded (e.g., it is not supporting any of the user's weight), and second extending the distraction assembly such that a small gap (e.g., approximately V) is created between the user's foot and a footplate of the foot cuff.

A second securing operation 730 secures the foot cuff to the user's hindfoot and/or midfoot. Specifically, with the device still unloaded (e.g., it is not supporting any of the user's weight), a second tightening mechanism may be retracted such that it pulls associated heel and forefoot straps downward, thereby pulling the user's foot away from the user's calf and downward toward the foot plate. This retraction is continued until the gap between the user's foot and the foot plate is closed. The second securing operation 730 may be accomplished using a second tightening mechanism that may be mechanical or electro-mechanical, and manual, partially-automated, or fully-automated, for example.

The foregoing sequence may be advantageous in that it gives an opportunity to measure how much force (e.g., distraction or offloading force) is required to return the foot to the footplate (close the gap). It also allows for a measurement of travel of the foot back to the foot plate to close the gap, which informs how much space might be created in the user's ankle joint. It also allows for further failsafe measures, for example, to release the distraction assembly if an unsafe condition is reached. In other implementations, the second securing operation 730 is performed prior to the extension operation 725 with similar effect.

An adjusting operation 735 adjusts the extension of the distraction assembly over time to accommodate varying conditions, such as the user's level of pain and state of activity. In a manual setting, the adjusting operation 735 is performed manually by the user when desired. In an automatic setting, the adjusting operation 735 is performed by the device constantly or iteratively in response to various inputs that indicate a change is distraction force is justified.

The presently disclosed technology may be implemented as logical steps in one or more computer systems (e.g., as a sequence of processor-implemented steps executing in one or more computer systems and as interconnected machine or circuit modules within one or more computer systems), such as computing system 600. The implementation is a matter of choice, dependent on the performance requirements of the computer system implementing the presently disclosed technology. Accordingly, the logical operations making up implementations of the presently disclosed technology are referred to variously as operations, steps, objects, or modules. Furthermore, the logical operations may be performed in any order, adding or replacing operations as desired, unless explicitly claimed otherwise or a specific order is inherently necessitated by the claim language.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

1. An ankle joint distraction device comprising:
a calf cuff including a calf tightening mechanism to selectively secure the calf cuff to a user's calf;
a foot cuff including a foot tightening mechanism to selectively secure to the user's foot, the foot cuff including a footplate to underlie the user's foot; and
a distraction assembly having a first distal end pivotally connected to the foot cuff and a second distal end fixedly connected to the calf cuff, the distraction assembly positioned between the foot cuff and the calf cuff, the distraction assembly to distract the user's ankle joint, a distraction force applied by the distraction assembly to vary based on the user's state of activity but maintain the user's foot in contact with the footplate.

2. The ankle joint distraction device of claim 1, wherein the calf cuff includes one or more rigid cuff sections to be form-fitted to the user's calf.

3. The ankle joint distraction device of claim 1, wherein the calf tightening mechanism is ratcheting with force multiplication.

4. The ankle joint distraction device of claim 1, wherein the foot cuff includes one or more rigid cuff sections.

5. The ankle joint distraction device of claim 4, wherein the foot cuff includes a heel strap and a bridge strap, in combination with the rigid cuff sections and the distraction force, the foot cuff to secure the user's foot within the foot cuff in all directions.

6. The ankle joint distraction device of claim 1, wherein the foot tightening mechanism is ratcheting with force multiplication.

7. The ankle joint distraction device of claim 1, wherein the distraction assembly includes a pair of linear ratchet mechanisms, each with a first distal end pivotally connected to the foot cuff and a second distal end fixedly connected to the calf cuff.

8. The ankle joint distraction device of claim 7, wherein each of the pair of linear ratchet mechanisms includes a ratcheting macro-adjuster to drive movement of a moveable linear rack with teeth in a singular linear direction and lock the moveable linear rack in position at tooth boundaries.

9. The ankle joint distraction device of claim 8, wherein each of the pair of linear ratchet mechanisms includes a ratcheting micro-adjuster to drive movement of the moveable linear rack in the singular linear direction and lock the moveable linear rack in position within its backlash between tooth boundaries.

10. An ankle joint distraction device comprising:
a calf cuff including a calf tightening mechanism to selectively secure the calf cuff to a user's calf;
a foot cuff including a foot tightening mechanism to selectively secure to the user's foot, the foot cuff including a footplate to underlie the user's foot;
a distraction assembly having a first distal end pivotally connected to the foot cuff and a second distal end fixedly connected to the calf cuff, the distraction assembly positioned between the foot cuff and the calf cuff, the distraction assembly to distract the user's ankle joint, the distraction force to vary based on the user's state of activity but maintain the user's foot in contact with the footplate;
one or more sensors to measure the distraction force; and
a microcontroller to vary a length of the distraction assembly based on the measured distraction force.

11. The ankle joint distraction device of claim 10, further comprising:
one or more sensors to measure compression in one or both of the calf cuff and the foot cuff, the microcontroller further to actuate one or both of the calf tightening mechanism and the foot tightening mechanism based on the measured compression.

12. The ankle joint distraction device of claim 10, wherein the distraction assembly is motor controlled in response to direction from the microcontroller.

13. The ankle joint distraction device of claim 10, wherein the sensors further include biometric sensors to monitor health of the user's leg.

14. The ankle joint distraction device of claim 10, wherein data collected by the sensors is uploaded to a computing network.

15. A linear ratchet mechanism comprising:
a moveable linear rack with teeth including a first attachment point at its distal end;
a spring-loaded pawl to engage the teeth and permit movement of the linear rack in a singular linear direction;
a mechanism housing enclosing the spring-loaded pawl, the linear rack including the first attachment point extending out of a first end of the mechanism housing, a second end of the mechanism housing includes a second attachment point; and
a ratcheting micro-adjuster to drive movement of the linear rack in the singular linear direction and lock the linear rack in position.

16. The linear ratchet mechanism of claim 15, wherein the ratcheting micro-adjuster includes:
a cable including distal ends attached to a rotating handle, the rotating handle to selectively shorten a length of the cable; and
a lifting hook attached to the linear rack, the cable to selectively lift the lifting hook in the singular linear direction when shortened.

17. The linear ratchet mechanism of claim 15, wherein the singular linear direction is extension of the first attachment point from the second attachment point.

18. The linear ratchet mechanism of claim 15, used to create joint distraction.

19. The linear ratchet mechanism of claim 15, wherein the spring-loaded pawl includes a release tab to disengage the spring-loaded pawl from the linear rack and permit movement of the linear rack in a direction opposite of the singular linear direction.

20. The linear ratchet mechanism of claim 15, wherein the mechanism housing includes a user-selectable preset for returning the first attachment point to a prior position with reference to the second attachment point.
